(19) United States
(12) Patent Application Publication
(10) Pub. No.: US 2023/0137160 A1
(43) Pub. Date: May 4, 2023

(54) LARGE SCALE PROCESS FOR PREPARING 1,2,4, 6-TETRA-O-ACETYL-3-AZIDO-3-DEOXY-D-GALACTOPYRANOSIDE

(71) Applicant: GALECTO BIOTECH AB, COPENHAGEN (DK)

(72) Inventors: Ulf NILSSON, Lund (SE); Jonathan CUMMINS, COPENHAGEN (DK)

(73) Assignee: GALECTO BIOTECH AB, COPENHAGEN (DK)

(21) Appl. No.: 17/915,690

(22) PCT Filed: Mar. 29, 2021
(86) PCT No.: PCT/EP2021/058120

§ 371 Date: Sep. 29, 2022

(30) Foreign Application Priority Data
Mar. 31, 2020 (EP) 20167113.8

(51) Int. Cl. C07H 13/04; C07H 1/00

(52) U.S. Cl. CPC C07H 13/04; C07H 1/00

(57) ABSTRACT

A process for preparing a compound of formula (X)

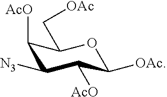

The process includes reacting a compound of formula (IX)

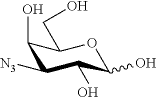

with an acetylating agent in the presence of a base in a suitable solvent and at a suitable temperature for a sufficient reaction time for preparing the compound of formula X. The process is suitable for large scale synthesis.

### TECHNICAL FIELD

The present invention relates to a process of preparing 1,2,4,6-Tetra-O-acetyl-3-azido-3-deoxy-α/β-D-galactopyranoside, in particular 1,2,4,6-Tetra-O-acetyl-3-azido-3-deoxy-β-D-galactopyranoside which process can be upscaled. The process parameters are stable, and the process is suitable for GMP manufacture.

### BACKGROUND ART

Idiopathic pulmonary fibrosis (IPF) represents a massive worldwide health burden. It is a chronic condition of unknown etiology in which repeated acute lung injury causes progressive fibrosis resulting in destruction of lung architecture, deteriorating lung function with consequent respiratory failure and death. Although idiopathic pulmonary fibrosis (IPF) is the arche-type and most common cause of lung fibrosis, numerous respiratory diseases can progress to pulmonary fibrosis, and this usually signifies a worse prognosis. The median time to death from diagnosis is 2.5 years and the incidence and prevalence of IPF continues to rise. It remains one of the few respiratory conditions for which there are no effective therapies, and there are no reliable biomarkers to predict disease progression. The mechanisms resulting in pulmonary fibrosis are unclear but center around aberrant wound healing as a consequence of repetitive epithelia injury from an as yet unknown cause. IPF is characterized by fibroblastic foci containing fibroblasts/myofibroblasts which show increased activation response to fibrogenic cytokines such as transforming growth factor-β1 (TGF-β1). There is a big unmet need for drugs for treatment of Idiopathic pulmonary fibrosis. The compound 3,3′-Dideoxy-3,3′-bis-[4-(3-fluorophenyl)-1H-1,2,3-triazol-1-yl]-1,1′-sulfanediyl-di-β-D-galactopyranoside is currently in clinical phase II for treatment of IPF and a process of preparing the compound is described in WO2014/067986. The compound of formula X is a highly important intermediate for preparing the above compound and consequently a process of making said compound which can be up scaled is needed.

### SUMMARY OF THE DISCLOSURE

The present invention relates to a new process for preparing 1,2,4,6-Tetra-O-acetyl azido-3-deoxy-β-D-galactopyranoside

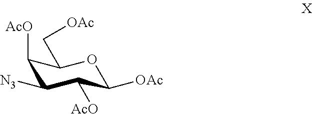

which process can be upscaled to large scale and/or industrial scale such as 30 kg or more, for instance 80 kg or more. For instance, from 2 kg to 80 kg, such as from 4 kg to 80kg, or from 10kg to 100kg. The process can also be used for smaller scale such as from 200 g to 2 kg.

In a first aspect the present invention relates to a process, such as suitable for large scale synthesis, for preparing a compound having formula (X)

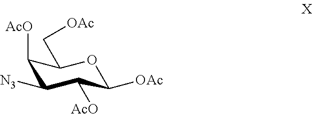

wherein the process comprises reacting a compound of formula IX

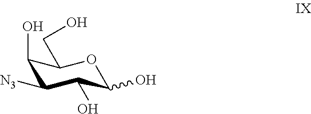

with an acetylating agent in the presence of a base in a suitable solvent and at a suitable temperature for a sufficient reaction time for preparing the compound of formula X.

In an embodiment the compound of formula X is purified and isolated as a solid. Preferably 1,2,4,6-Tetra-O-acetyl-3-azido-3-deoxy-β-D-galactopyranoside is isolated as a white solid, such as crystalline or amorphous.

In a further embodiment the acetylating agent is selected from one or more of acetic anhydride; acyl chloride; acetic acid in the presence of an activating agent such as carbonyl diimidazole or a dialkyl carbodiimide; acid catalysis under dehydrating conditions; or transesterification using an acyl ester.

In a still further embodiment the base is selected from one or more of tertiary amines, such as triethylamine or diisoproylethylamine; or an aromatic amine base, such as pyridine or imidazole, optionally in the presence of a catalytic stronger base such as dimethylaminopyridine.

In a further embodiment the suitable solvent is selected from one or more cyclic or acyclic ethereal solvents, such as 1,4-dioxane, 2-methyl tetrahydrofuran or tertiary butyl methyl ether; an ester solvent, such as ethyl acetate or isopropyl acetate; an aromatic solvent such as toluene.

In a still further embodiment the suitable temperature is from -5° C. to 40° C. Typically from −5° C. to 35° C.

In a further embodiment the addition of the acetylating agent optionally in the suitable solvent is performed over a period of at least 30 minutes, such as at least 3 hours.

In a still further embodiment the reaction time is from 1 to 24 hours.

In a further embodiment the process comprises a preceding step for preparing the compound having formula IX wherein the preceding step comprises reacting a compound of formula VIII

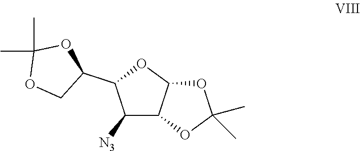

with an acid, such as an acidic cation exchange resin or p-toluene sulfonic acid, in a suitable solvent at a suitable temperature for a sufficient reaction time, for preparing the compound of formula IX.

In an embodiment the acid is selected from one or more acidic cation exchange resin, such as Amberlite IR-120 H, dilute hydrochloric acid, or p-toluene sulfonic acid.

In a further embodiment the suitable solvent is a mixture of an organic solvent and water, such as 1,4-dioxane, 2-methyl tetrahydrofuran, tetrahydrofuran, or acetonitrile and water.

In a still further embodiment the suitable temperature is from 25° C. to 70° C.

In a further embodiment the reaction time is 1 to 24 hours.

In a still further embodiment the process comprises a preceding step for preparing the compound having formula VIII wherein the preceding step comprises reacting a compound of formula VII

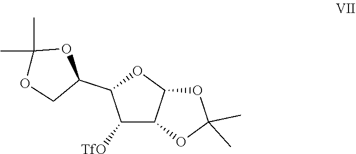

with a suitable azide in a suitable solvent at a suitable temperature for a sufficient reaction time, for preparing the compound of formula VIII.

In an embodiment the azide is selected from an azide salt, such as sodium azide or potassium azide.

In a further embodiment the suitable solvent is selected from one or more dipolar aprotic solvents, such as dimethylformamide, dimethylsulfoxide or acetonitrile; or biphasic systems, such as tertiary butyl methyl ether or similar water immiscible solvents, such as 2-methyl tetrahydrofuran/water with a phase transfer catalyst, such as tetrabutylammonium bromide.

In a still further embodiment the suitable temperature is from 0° C. to 30° C.

In a further embodiment the reaction time is 30 min to 22 hours.

In a still further embodiment the process comprises a preceding step for preparing the compound having formula VII wherein the preceding step comprises reacting a compound of formula VI

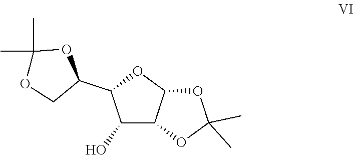

with a suitable base in a suitable solvent followed by a trifluoromethylating agent in a suitable solvent at a suitable temperature for a sufficient reaction time, for preparing the compound of formula VII.

In an embodiment the suitable base is pyridine or a hindered aliphatic tertiary amine, such as diisopropylethylamine.

In a further embodiment the triflating agent is selected from one or more trifluoro-methanesulfonic anhydride or an equivalent triflating agent, such as N-phenyl-bis(trifluoromethanesulfonimide).

In a still further embodiment the suitable solvent is independently selected from an aprotic solvent, such as tertiary butyl methyl ether, toluene or tetrahydrofuran.

In a further embodiment the suitable temperature is from 0° C. to 30° C. 23. The process of any one of claims 19-22 wherein the suitable temperature is from -5° C. to 30° C.

In a still further embodiment the reaction time is at least 1 hour.

### DETAILED DESCRIPTION

The compound of formula (X) has the chemical name (IUPAC) 1,2,4,6-Tetra-O-acetyl-3-azido-3-deoxy-β-D-galactopyranoside. The compound of formula X is the beta anomer however the mixture of alpha and beta anomers have been disclosed in Lowary, T. L. and Hindsgaul, O. (1994) Recognition of synthetic O-methyl, epimeric, and amino analogues of the acceptor alpha-L-Fucp-(1,2-beta-D-Galp-OR by the blood group A and B gene-specified glycosyltransferases. Carbohydr. Res. 251: 33-67. The alpha and beta anomers may be separated by various methods such as via crystallization. However, for the present process the preferred aim is to prepare the beta anomer.

Further embodiments of the process are described in the experimental section herein, and each individual process as well as each starting material constitutes embodiments that may form part of embodiments.

The above embodiments should be seen as referring to any one of the aspects (such as ‘method for treatment’, ‘pharmaceutical composition’, ‘compound for use as a medicament’, or ‘compound for use in a method’) described herein as well as any one of the embodiments described herein unless it is specified that an embodiment relates to a certain aspect or aspects of the present invention. All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### Experimental

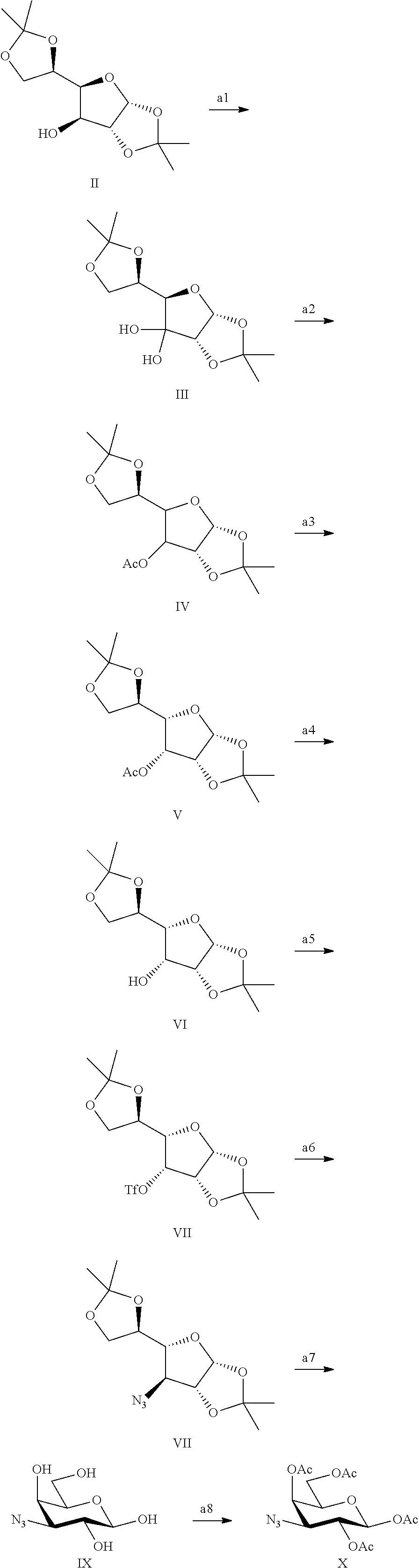

The current process to manufacture the compound having formula X involves several process steps as described in detail hereunder.

General Procedures

Nuclear Magnetic Resonance (NMR) spectra were recorded on a 400 MHz Bruker Avance AV400 spectrometer at 25° C. Chemical shifts are reported in ppm (δ) using the residual solvent as the internal standard. Peak multiplicities are expressed as follows: s, singlet; d, doublet; t, triplet; q, quartet; m, multiplet; br s, broad singlet.

The following abbreviations are used:

Ac: Acetyl

aq.: aqueous

DCM: Dichloromethane

DMF: N,N-Dimethylformamide

rt: room temperature

Sat.: Saturated

TBME: tert-Butylmethyl ether

TEMPO: (2,2,6,6-Tetramethylpiperidin-1-yl)oxyl

### 1,2:5,6-di-O-Isopropylidene-α-D-glucofuran-3-ulose hydrate, III

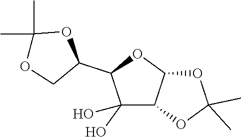

To 1,2:5,6-di-O-isopropylidene-α-D-glucofuranose, II (3.5 kg, 13.45 mol) solubilized in a mixture of ethyl acetate (17.33 L) and water (7.00 L) at 20° C. was added NaBr (1.08 kg, 10.5 mol), NaOAc (1.66 kg, 20.18 mol) and TEMPO (21 g, 0.13 mol). The reaction mixture was cooled from 20° C. to 12° C. over 30 minutes and a solution of sodium hypochlorite (14%, 12.9 L, 29.59 mol) was added. The mixture was stirred for 30 minutes at 18° C. then 2M aq. Na₂S₂O₃ was added until a negative starch-iodide test was observed. The phases were separated, and the organic phase was washed with sat. aq. NaCl solution (3.5 L), dried over MgSO₄ and concentrated in vacuo using a rotary evaporator to give 3.72 kg of 1,2:5,6-di-O-isopropylidene-α-D-glucofuran-3-ulose hydrate as an oily solid. ¹H NMR (400 MHz, CDCl₃) δ 5.86 (d, J=3.8 Hz, 1H), 4.43 (q, J=6.3 Hz, 1H), 4.26 (d, J=3.8 Hz, 1H), 4.15 (dd, J=8.7, 6.3 Hz, 1H), 4.09 (dd, J=8.7, 6.1 Hz, 1H), 3.97 (br s, 1H), 3.91 (d, J=6.5 Hz, 1H), 3.62 (br s, 1H), 1.58 (s, 3H), 1.50 (s, 3H), 1.38 (s, 3H), 1.36 (s, 3H).

### Alternative Procedure

To 1,2:5,6-di-O-isopropylidene-α-D-glucofuranose, II (112 kg, 430 mol) solubilized in a mixture of ethyl acetate (504 L) were added demineralized water (168 L), NaBr (35.0 kg, 340 mol), NaOAc (53.0 kg, 646 mol) and further demineralized water (56 L) at 27° C. The reaction mixture was cooled to 0° C. then TEMPO (1.0 kg, 6.45 mol) and further ethyl acetate (56 L) added. To a separate vessel sodium hypochlorite (9-12% w./w, 1000 L, ca. 1649 mol) was added and the pH adjusted to 11.5 to 12.5 with a 1:1 mixture of concentrated sulphuric acid:water (ca. 67 L) at 2.5° C. The sodium hypochlorite solution was filtered and added to the reaction mixture at 7° C. over 7.5 hours. The mixture was stirred for 1 hour at 3° C. then 20% w/w aq. sodium sulfite (135 kg) was added at 3° C. The mixture was warmed to 29° C. and the phases separated. The organic phase was checked for absence of sodium hypochlorite. To the aqueous phase, solid NaCl (56 kg) was added, and then it was extracted with ethyl acetate (225 L). To the aqueous phase solid NaCl (28 kg) was added, and then it was extracted with ethyl acetate (224 L). The combined organic phases were washed with 9% w/w aqueous sodium chloride (112 kg) at 30° C., the layers separated and the aqueous layer back-extracted with ethyl acetate (112 L). The combined organic layers were dried with MgSO₄ (56 kg), filtered, the filter cake washed with ethyl acetate (56 L), and the combined filtrates were concentrated in vacuo at up to 45° C. to 392 L. Three further ethyl acetate addition (225, 225 and 112 L), distillation cycles were performed to control water to ≤0.3% w/w and the assay of the solution determined. 1,2:5,6-di-O-isopropylidene-α-D-glucofuran-3-ulose hydrate III was progressed to the next step of the process as an ethyl acetate solution with an assumed 119 kg, 100% th yield.

### 3-O-Acetyl-1,2:5,6-di-O-isopropylidene-α-D-erythro-hex-3-enofuranose, IV

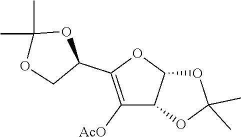

To a solution of 1,2:5,6-di-O-isopropylidene-α-D-glucofuran-3-ulose hydrate, III (3.5 kg, 12.7 mol) in pyridine (7.17 L, 88.7 mol) at 20° C. was charged acetic anhydride (3.60 L, 38.1 mol) portionwise. The reaction mixture was heated to 60° C. and stirred for 16 hours. The mixture was cooled to 20° C. and TBME (14 L) was charged. The mixture was washed sequentially with 2M aq. HCl solution (2×10.5 L), water (7 L), 5% aq. NaHCO₃ solution (10.5 L) and sat. aq. NaCl solution (7 L) at 20° C.+/−5° C. The organic phase was dried over MgSO₄ and concentrated in vacuo using a rotary evaporator to give 2.78 kg of 3-O-acetyl-1,2:5,6-di-O-isopropylidene-α-D-erythro-hex-3-enofuranose as an off-white solid. ¹H NMR (400 MHz, CDCl₃) δ 5.95 (d, J=5.5 Hz, 1 H), 5.31 (d, J=5.5 Hz, 1H), 4.62 (t, J=6.4 Hz, 1H), 4.03-3.94 (m, 2H), 2.12 (s, 3H), 1.45 (s, 3H), 1.39 (s, 3H), 1.36 (s, 3H), 1.30 (s, 3H).

Alternative Procedure

To a solution of 1,2:5,6-di-O-isopropylidene-α-D-glucofuran-3-ulose hydrate III (119 kg, 430 mol) in ethyl acetate (ca. 301 L progressed from previous step) was added pyridine (85.1 kg, 1075 mol), dimethylamino pyridine (DMAP) (11.2 kg, 91.7 mol), ethyl acetate (112 L) and acetic anhydride (88 kg, 862 mol) at 32° C. The reaction was heated to 60° C. for 4 hours then cooled to 27° C. and methanol (11.2 L) added. The mixture was stirred at 27° C. for 45 minutes, cooled to 12° C., ethyl acetate (675 L) added then the organic solution washed with 2M aqueous HCl (3×336 L) at 15° C. The organic layer was washed with water (340 L), 5% aqueous NaHCO₃ solution three times (560 L then 2×340 L) then 5% aqueous NaCl (225 L) at 15° C. The organic layer was separated and concentrated to ca. 112 L, at up to 45° C., in vacuo, then n-heptane (225 L) added and distillation continued. Further heptane (170 L) was added and distillation continued to ca. 112 L, in vacuo, at up to 45° C. The residue was cooled to 25° C. and TBME (900 L) added. The solution was cooled to 10° C. and washed with 1M aqueous HCl (3×294 L), water (340 L), 5% w/w aqueous NaHCO₃ (3×560 L) then 5% w/w aqueous NaCl (225 L). The organic layer was separated and treated with Amberlite IR-96 resin (4.5 kg) whilst distilling to ca. 112 L, in vacuo, at up to 40° C. Methanol (900 L) was added to the residue and the mixture treated with activated charcoal (11.2 kg) at 30° C. The mixture was filtered over filter aid and 3-O-acetyl-1,2:5,6-di-O-isopropylidene-α-D-erythro-hex-3-enofuranose IV (93.6 kg, 72% th) was progressed to the next stage as a solution in methanol.

### 3-O-acetyl-1.2:5.6-di-O-Isopropylidene-α-D-gulofuranose, V

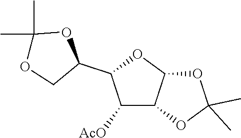

To a vessel containing 10% Pd/C (50% wet, 142 g, 0.067 mol) was charged solution of 3-O-acetyl-1,2:5,6-di-O-isopropylidene-α-D-erythro-hex-3-enofuranose, IV (1.0 kg, 3.33 mol) in methanol (7.5 L) at 20° C. A solution of triethylsilane (1.60 L, 9.99 mol) in methanol (5.25 L) was added dropwise maintaining the temperature less than 35° C. using a jacketed vessel. The mixture was stirred for 10 minutes at 20° C. then filtered through a short pad of celite® washing with methanol (1.5 L) to give a solution of 3-O-acetyl-1,2:5,6-di-O-isopropylidene-α-D-gulofuranose in methanol which was used directly in the production of 1,2:5,6-Di-O-isopropylidene-α-D-gulofuranose, VI.

### 1,2:5,6-di-O-Isopropylidene-α-D-gulofuranose, VI

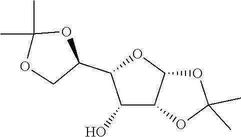

To a solution of 3-O-acetyl-1,2:5,6-di-O-isopropylidene-α-D-gulofuranose, V (3.33 mol) in methanol at rt was charged a solution of 25 wt% sodium methoxide in methanol to pH 11. The reaction mixture was stirred at rt for 1 hour, followed by the addition of solid carbon dioxide until the pH was ≤7.5. The mixture was concentrated in vacuo at 40° C. using a rotary evaporator and the crude residue was suspended in ethyl acetate (3.9 L) and heated to 60° C. The mixture was allowed to cool to ≤40° C. then filtered, washing with ethyl acetate (1.5 L). The mixture was concentrated in vacuo at 40° C. to a volume of 2.5 L and heated to 60° C. Heptane (4.65 L) was added and the mixture cooled from 60° C. to 5° C. and stirred for 30 minutes. The product was isolated by filtration washing with heptane (3×0.5 L). The filter cake was dried under vacuum at 40° C. to give 0.60 kg (47% over 4 steps from II) of 1,2:5,6-di-O-isopropylidene-α-D-gulofuranose as an off-white solid. ¹H NMR (400 MHz, CDCl₃) δ 5.78 (d, J=4.1 Hz, 1H), 4.66 (dd, J=6.3, 4.1 Hz, 1H), 4.52 — 4.43 (m, 1H), 4.26 — 4.19 (m, 2H), 3.90 (dd, J=8.6, 5.7 Hz, 1H), 3.72 (dd, J=8.6, 7.2 Hz, 1H), 2.64 (d, J=6.4 Hz, 1H), 1.63 (s, 3H), 1.45 (s, 3H), 1.43 (s, 3H),1.38 (s, 3H).

Alternative Procedure

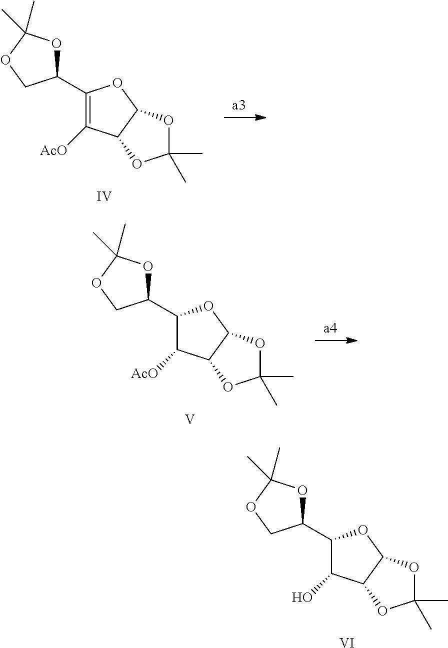

To a solution of solution of 3-O-acetyl-1,2:5,6-di-O-isopropylidene-α-D-erythro-hex-3-enofuranose, IV (79.9 kg, 266 mol) in methanol (ca. 874 L), Amberlite IRA-96 resin (11.2 kg), which had been pre-washed with water and methanol, was charged. The mixture was stirred for 20 minutes at 30° C. and the pH confirmed to be ≥6.0. The mixture was filtered, the filter bed washed with methanol (28 L) and 10% Pd/C (2.8 kg) charged. The reaction was placed under hydrogen at 5 to 6 bar at 33° C. for 1 h then filtered, the filter cake washed with methanol (225 L). The filtrates were distilled to ca. 560 L in vacuo at up to 45° C. To the residue, 30% sodium methoxide in methanol (4.5 L) was charged to adjust the pH to ≥11.0 and the mixture stirred for 2.5 hours at 28° C. Solid carbon dioxide was added in 5.6 kg portions to adjust the pH to ≤7.5, and the reaction mixture distilled to ca. 224 L in vacuo at up to 45° C. Ethyl acetate (3×340 L) was added and distilled in vacuo at up to 45° C. to ca. 224 L. Ethyl acetate (225 L) was added to the residue and the methanol level confirmed to be ≤2.0%. The solution was treated with activated charcoal (28 kg) at 44° C. for 20 minutes then filtered through filter aid and the filter washed with ethyl acetate (225 L). The combined filtrates were distilled to ca. 120 L in vacuo at up to 45° C. and heptane (790 L) added at 45° C. The mixture was cooled to −2° C., held at that temperature for 1 hour then filtered and the filter cake washed with heptane at 0° C. The 1,2:5,6-di-O-isopropylidene-α-D-gulofuranose was dried at up to 40° C. in vacuo to deliver 50.9 kg, 45% over four steps from II.

### 1,2:5,6-di-O-Isopropylidene-3-O-trifluoromethanesulfonate-α-D-gulofuranose, VII

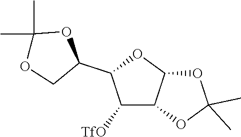

To a solution of 1,2:5,6-di-O-isopropylidene-α-D-gulofuranose, VI (0.66 kg, 2.54 mol) in TBME (3.3 L) at rt was added pyridine (493 mL, 6.10 mol). The suspension was cooled to 0° C. Trifluoromethanesulfonic anhydride (513 mL, 3.05 mol) was added dropwise to the mixture maintaining the temperature less than 15° C. The reaction mixture was stirred at 10° C. for 1 hour then washed successively with 2M aq. HCl solution (1.32 L), 5% aq. NaHCO₃ solution (2×1.32 L) and 10% aq. NaCl solution (1.32 L). DMF (4.95 L) was added to the organic phase at 20° C. +/−5° C. and the TBME removed by concentration in vacuo at 40° C. using a rotary evaporator to give a solution of 1,2:5,6-Di-O-isopropylidene-3 trifluoromethanesulfonate-α-D-gulofuranose in DMF which was used directly in the production of 3-Azido-3-deoxy-1,2:5,6-di-O-isopropylidene-α-D-galactofuranose, VIII.

### 3-Azido-3-deoxy-1,2:5,6-di-O-isopropylidene-α-D-galactofuranose, VIII

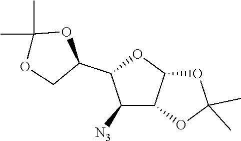

A solution of 1,2:5,6-di-O-isopropylidene-3-O-trifluoromethanesulfonate-α-D-gulofuranose, VII (2.54 mol) in DMF (4.95 L) was cooled to 0° C. and NaN₃ (330 g, 5.08 mol) was added portionwise. The mixture was stirred at 5° C. for 30 minutes then warmed to 20° C. and stirred for 16 hours. Water (3.6 L) was added dropwise maintaining the temperature in the range 20° C.±5° C. and the mixture was extracted with TBME (2×1.92 L). The combined extracts were washed sequentially with 5% aq. NaHCO₃ solution (3.6 L) and 10% aq. NaCl solution (3.6 L). The organic phase was dried over MgSO₄ and concentrated in vacuo at 40° C. to give 655 g (91% over two steps from VI) of 3-azido-3-deoxy-1,2:5,6-di-O-isopropylidene-α-D-galactofuranose. ¹H NMR (400 MHz, CDCl₃) δ 5.70 (d, J=4.0 Hz, 1H), 4.50 (dd, J=3.9, 1.8 Hz, 1H), 4.25 (td, J=6.7, 5.7 Hz, 1H), 3.98 (dd, J=8.4, 6.7 Hz, 1H), 3.85 (dd, J 5.6, 1.8 Hz, 1H), 3.78 (dd, J=8.4, 6.7 Hz, 1H), 3.73 (t, J=5.6 Hz, 1H), 1.48 (s, 3H), 1.36 (s, 3H), 1.29 (s, 3H), 1.29 (s, 3H).

Alternative Procedure

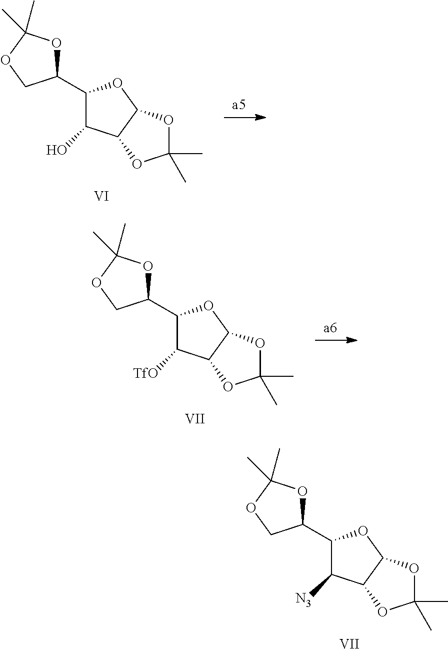

To a solution of 1,2:5,6-di-O-isopropylidene-α-D-gulofuranose, VI (45 kg, 173 mol) in TBME (225 L) pyridine (41.0 kg, 519 mol) was added and the solution cooled to 1° C. Trifluoromethanesulfonic anhydride (73.1 kg, 259 mol) was added slowly to the suspension, maintaining 0° C., followed by TBME (45 L). The reaction mixture was warmed to 15° C., stirred for 1.5 hours then cooled to 5° C. and water (225 L) added at ≤10° C. The mixture was warmed to 18° C. and the layers separated. The aqueous phase was extracted with TBME (180 L), the combined organic layers washed with 5% aqueous NaHCO₃ solution (135 L) then 5% aqueous NaCl solution (135 L). Solvent exchange to DMF was performed through distillation in vacuo at up to 35° C. and the resulting solution (ca. 270 L) cooled to 20° C. The pH was adjusted to ≥8.0 with triethylamine and the solution cooled to 1° C. prior to addition of sodium azide (22.5 kg, 346 mol) in 5 portions over 1 hour. The temperature was increased to 18° C. and the mixture stirred for 3 hours. The reaction was cooled to 5° C. and water (450 L) added at 10° C. The mixture was extracted with TBME (2×180L) and the combined organic extracts washed with 5% aqueous NaHCO₃ solution (135 L), then water (135 L). The organic layer was separated and solvent exchange to 1,4-dioxane performed by distillation in vacuo, at up to 50° C. 3-azido-3-deoxy-1,2:5,6-di-O-isopropylidene-α-D-galactofuranose VIII (43 kg, 88% over two steps from VI) was progressed to the manufacture of 1,2,4,6-Tetra-O-acetyl-3-azido-3-deoxy-β-D-galactopyranose X, as a solution in 1,4-dioxane, ca. 90 L total volume.

Alternative Process

To a solution of 1,2:5,6-di-O-isopropylidene-α-D-gulofuranose, VI (200 g, 0.77 mol) in TBME (540 mL) pyridine (112 mL, 1.84 mol) was added and the solution cooled to 5° C. Trifluoromethanesulfonic anhydride (262 g, 0.93 mol) was added over 2 hours to the suspension maintaining 5° C. followed by TBME (72 mL). The reaction mixture was warmed to 10° C. and stirred for 1 hour then quenched by addition of sodium bisulphite solution [made from pre-mixed 50% aqueous NaOH (22.0 g), water (138 mL) and 95% sulphuric acid (56 g)] at 5° C. Water (140 mL) was added and the mixture warmed to 20° C. The layers were separated and the organic phase washed with water (140 mL), 5% aqueous NaHCO₃ solution (140 mL) then 18% aqueous NaCl solution (128 mL) at 20° C. The layers were separated and triethylamine hydrochloride (80 g, 0.58 mol) and tetrabutyl ammonium bromide (TBAB) (24.0 g, 0.16 mol) added to the organic phase. Sodium azide (74 g, 1.14 mol) dissolved in water (200 mL) was added at 20° C. then the mixture heated to 50° C. The reaction was held for 20 hours then cooled to 20° C. and water (400 mL) added. The phases were separated and the organic phase washed with water (200 mL) then 18% aqueous sodium chloride (120 mL). A solvent exchange to 2-methyl tetrahydrofuran (2-MeTHF)was performed through distillation in vacuo at up to 60° C. 3-azido-3-deoxy-1,2:5,6-di-O-isopropylidene-α-D-galactofuranose VIII (206 g, 94% th over two steps from VI) was progressed to the manufacture of 1,2,4,6-Tetra-O-acetyl-3-azido-3-deoxy-β-D-galactopyranose X, as a solution in 2-MeTHF, ca. 840 mL total volume.

### 3-Azido-3-deoxy-D-galactopyranose, IX

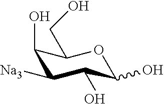

A mixture of 3-azido-3-deoxy-1,2:5,6-di-O-isopropylidene-α-D-galactofuranose, VIII (655 g, 2.30 mol) dissolved in 1,4-dioxane (2.62 L), water (0.65 L) and a strong acidic cation exchange resin (262 g) was stirred at 60° C. for 18 hours. The mixture was cooled to 20° C. and filtered, washing the resin with 4:1 1,4-dioxane:water (1.96 L). To the filtrate solution at rt was charged a strong acidic cationic/weak basic anionic mixed bed resin (262 g) and the mixture stirred at rt for 1 hour. The mixture was filtered, washing the resin with 4:1 1,4-dioxane:water (1.96 L). The filtrate was concentrated in vacuo at 40° C. using a rotary evaporator and co-evaporated with acetonitrile (2×0.66 L) to give 468 g (99%) of 3-azido-3-deoxy-D-galactopyranose as a white solid. ¹H NMR (400 MHz, CDCl₃) δ 5.16 (d, J=3.8 Hz, 1H), 4.03 (t, J=6.2 Hz, 1H), 4.00 — 3.94 (m, 2H), 3.70 — 3.66 (m, 2H), 3.60 (dd, J=10.7, 3.0 Hz, 1H).

### 1,2,4,6-Tetra-O-acetyl-3-azido-3-deoxy-β-D-galactopyranose, X

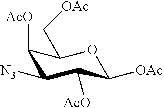

To a suspension of 3-azido-3-deoxy-D-galactopyranose, IX (770 g, 3.75 mol) in ethyl acetate (2.31 L) and triethylamine (6.27 L, 45.0 mol) was added a solution of acetic anhydride (3.54 L, 37.5 mol) in ethyl acetate (1.54 L) dropwise maintaining the temperature ≤35° C. The reaction mixture was stirred for 18 hours at 30° C. then cooled to 0° C. The mixture was washed successively with 2M aq. HCl solution (5.0 L), water (1.54 L), 5% aq. NaHCO₃ solution (1.54 L) and 10% aq. NaCl solution (1.54 L) maintaining the temperature ≤25° C. The organic phase was concentrated in vacuo, and the residue was suspended in ethyl acetate (2.0 L) at room temperature. The suspension was warmed to 60° C. until complete dissolution of material. The mixture was cooled to 20° C. and heptane (2.89 L) was charged. The mixture was cooled to 0° C., stirred for 1 hour then filtered, washing the solid with 4:1 heptane:ethyl acetate (0.77 L). The solid was dried under vacuum at 50° C. to give 882 g (63%) of 1,2,4,6-tetra-O-acetyl-3-azido-3-deoxy-β-D-galactopyranose as a white solid. ¹H NMR (400 MHz, CDCl₃) δ 5.70 (d, J=8.3 Hz, 1H), 5.48 (dd, J=3.4, 0.9 Hz, 1H), 5.30 (dd, J=10.6, 8.3 Hz, 1H), 4.23-3.99 (m, 3H), 3.69 (dd, J=10.6, 3.4 Hz, 1H), 2.20 (s, 3H), 2.14 (s, 3H), 2.14 (s, 3H), 2.07 (s, 3H).

Alternative Procedure

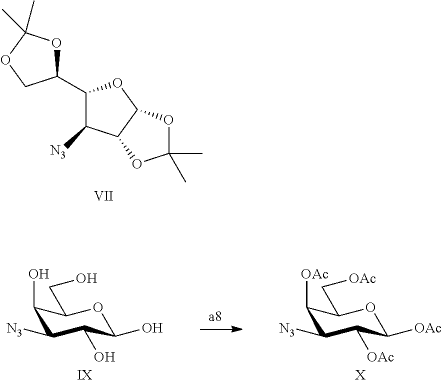

A solution of 3-azido-3-deoxy-1,2:5,6-di-O-isopropylidene-α-D-galactofuranose, VIII (43.4 kg, 152 mol) in 1,4-dioxane (ca. 90 L total volume) was charged to a vessel followed by 1,4-dioxane (44 L), activated Amberlite IR-120 resin (12.3 kg), and water (87.2 L). The mixture was heated to 62° C. for 24 hours then cooled to 32° C. and filtered. The filter was washed with further 1,4-dioxane (105 L). The filtrates were treated with activated Amberlite IRA-96 resin (23 kg) for 1.5 hours at 30° C. to adjust the pH to ≥6.0. The mixture was filtered and the filter cake washed with 1,4-dioxane (105 L). Water was removed from the combined filtrates through addition and distillation of 1,4-dioxane (3×217 L), in vacuo, at up to 50° C. resulting in a reaction volume of ca. 107 L. The reaction was cooled to 32° C. and triethylamine (216 kg, 2132 mol) and 1,4-dioxane (129 L) added. Acetic anhydride (157 kg, 1533 mol) in 1,4-dioxane (44 L) was charged to the vessel over 5 hours at 32.5° C. and the mixture stirred at 30° C. for 22 hours. Dimethylamino pyridine (DMAP) (4.34 kg, 35.5mo1) was charged followed by acetic anhydride (15.5 kg, 152 mol) in 1,4-dioxane (11 L) over 39 minutes at 30° C. and the mixture stirred at 30° C. for 4 hours. The reaction was cooled to 10° C. and water (1032 L) added. The mixture was filtered and the filter cake washed with water (84 L). The wet cake was dissolved in ethyl acetate (304 L) and treated with activated carbon (8.7 kg) for 1 hour. The mixture was filtered and the filter cake washed with ethyl acetate (87 L). The combined filtrates were washed with 10% aqueous NaCl (239 kg) at 20° C. The layers were separated and the organic layer was distilled to low volume, in vacuo, at up to 45° C. Diisopropyl ether (DIPE) (93 L) was added over 30 minutes at 35 to 45° C. The mixture was stirred at 28° C. for 1 hour then n-heptane (259 L) added at 24° C. over 30 minutes. The resulting slurry was cooled to −2° C. and stirred for 1.5 hours. The mixture was filtered, the filter cake washed with cold n-heptane (43.4 L). The filter cake was dried in vacuo at up to 45° C. to deliver 1,2,4,6-tetra-O-acetyl-3-azido-3-deoxy-β-D-galactopyranose X (34.1 kg, 59.9% over two steps from VIII) as a white solid.

Alternative Procedure

To a solution of 3-azido-3-deoxy-1,2:5,6-di-O-isopropylidene-α-D-gulofuranose, VIII (206 g, 0.72 mol) in 2-MeTHF (ca. 900mL total volume) was added para-toluenesulphonic acid (27.3g, 0.16 mol) and water (104 mL). The mixture was heated to 50° C. and stirred for 18 hours. Triethylamine (21.2 mL) was added, and the solution dried by azeotropic distillation of 2-MeTHF in vacuo, at 40 to 50° C. The solution was cooled to 40° C. and triethylamine (906 mL, 6.5 mol) added. Acetic anhydride (513 mL, 5.4 mol) was added over 3 hours and the solution held at 40° C. for 18 hours. The reaction was cooled to 0° C. and further 2-MeTHF (474 mL) and water (618 mL) charged at 0° C. The mixture was warmed to 20° C., stirred for an hour and the phases separated. The organic phase was washed with water twice (2×618 mL) then further 2-MeTHF added (762 mL) prior to distillation in vacuo at 40 to 50° C. to ca. 1.5 L. The residual solution was treated with activated carbon (4.1 g) at 40° C. for 1.5 h, filtered, then distilled to ca 750 mL in vacuo at 40 to 50° C. The solution was cooled to 42° C. and seed crystals (2.1 g) added. The slurry was held at 42° C. for 3 hours, cooled slowly to 20° C. then cyclohexane (507 mL) added over 4 hours. The slurry was held for 2 hours then filtered and the filter cake washed with a mixture of 2-MeTHF:cyclohexane (95:371 mL). The product was dried in vacuo at up to 40° C. to give 1,2,4,6-Tetra-O-acetyl-3-azido-3-deoxy-β-D-galactopyranose (167 g, 62% over 2 steps from VIII) as a white solid.

Alternative Procedure

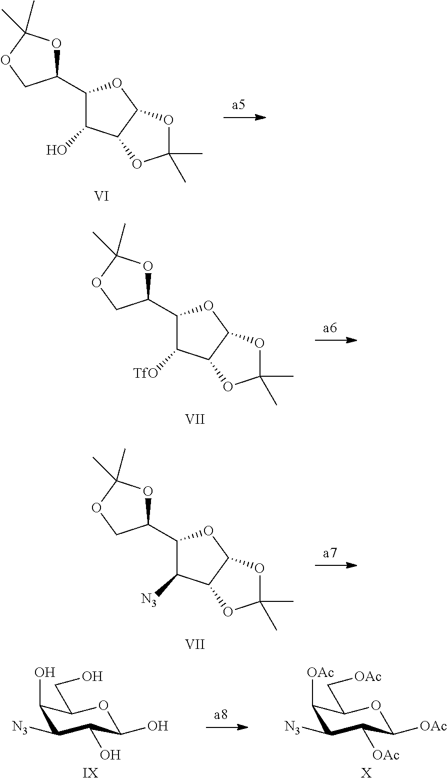

To a solution of 1,2:5,6-Di-O-isopropylidene-α-D-gulofuranose, VI (35 kg, 134 mol) in TBME (130 L) was added pyridine (31.9 kg, 403 mol) at 25° C. The suspension was cooled to 0° C. and trifluoromethanesulfonic anhydride (61.6 kg, 218 mol) added to the mixture at ≤20° C. over 1.5 hours. The reaction mixture was stirred at ≤20° C. for 15 minutes then warmed to 25° C. and stirred for 6 hours. The mixture was cooled to 15° C. then the pH adjusted to 3.0 with 1.5 M aq. HCl solution. The mixture was filtered, the filter cake washed with TBME (70 kg) and the phases were separated. The organic phase was washed with 5% aq. NaHCO₃ solution (73 kg) and 10% aq. NaCl solution (77 kg) at 25° C. The layers were separated and DMF (35 L) added to the organic phase. TBME was removed by concentration in vacuo at up to 35° C. and further DMF (133 kg) added to the residue. Distillation was continued to give a solution of 1,2:5,6-Di-O-isopropylidene-3-O-trifluoromethanesulfonate-α-D-gulofuranose (VII) in DMF (ca. 250 L). Triethylamine (0.25 kg) was added, and the solution cooled to 5° C. then NaN₃ (17.5 kg, 269 mol) added in portions over 1 hour. The mixture was stirred at 5° C. for 30 minutes, warmed to 25° C. and stirred for 1 hour. The reaction was cooled to 10° C. and water (263 kg) added slowly maintaining the temperature at 10° C. The mixture was extracted with TBME twice (130 kg then 106 kg) at 20° C. The combined organic extracts were washed sequentially with 5% aq. NaHCO₃ solution (126 kg) and 10% aq. NaCl solution (2×116 kg). The organic phase was concentrated in vacuo at up to 50° C. to ca. 100 L. 1,4-dioxane (74 kg) was added and distillation continued to ca. 100 L twice. The solution was cooled to 25° C. then Pre-washed Amberlite IR-120 resin (14 kg) and water (35 kg) were added. The mixture was heated to, and stirred at, 65° C. for 24 hours. The mixture was cooled to 25° C., filtered and the filter cake washed with 4:1 w/w 1,4-dioxane:water (105 kg). Pre-washed Amberlite IRA-96 resin (15.8 kg) was charged to the combined filtrates and the mixture stirred at 25° C. for 3 hours. Triethylamine (2.5 kg) was charged to adjust the pH to 6.5 to 7.5 and the mixture was filtered, washing the resin with 4:1 1,4-dioxane:water (105 kg). The combined filtrates were dried by concentration in vacuo at up to 65° C. to ca. 120 L followed by addition and distillation of 1,4-dioxane (3×84 kg). The residual solution was cooled to 25° C. and triethylamine (151 kg, 1495 mol) was added. Acetic anhydride (126 kg, 1234 mol) was added to the solution over 4 hours at 25° C. The reaction mixture was stirred for 24 hours at 30° C., cooled to 20° C. then water (228 kg) and 2-methyl tetrahydrofuran (14.7 kg) were added. The pH of the reaction mixture was adjusted to 1 to 2 with 1.5M aq. HCl solution (105 kg) at 5° C. The mixture was stirred at 5° C. for 2 hours then filtered and the filter cake washed with water (2×175 kg). The filter cake was slurried with aqueous potassium phosphate, dibasic (20% w/w, 175 kg) for 30 minutes at 25° C., then filtered, and the solids washed with water (174 kg). The solids were washed with n-heptane (47.6 kg) then dried at 35° C. The crude material was further slurried in water (255 kg) at 25° C. for 2 hours twice then dried at 35° C. to give 1,2,4,6-Tetra-O-acetyl-3-azido-3-deoxy-β-D-galactopyranose (24.4 kg, 49% over 4 steps from VI) as a white solid.

1-24. (canceled)

25. A process suitable for large scale synthesis for preparing a compound having formula (X) 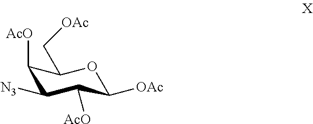
wherein the process comprises reacting a compound of formula IX 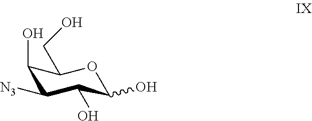
with an acetylating agent in the presence of a base in a suitable solvent and at a suitable temperature for a sufficient reaction time for preparing the compound of formula X.

26. The process of claim 25, wherein the compound of formula X is purified and isolated as a solid.

27. The process of claim 25, wherein the acetylating agent is selected from one or more of acetic anhydride; acyl chloride; acetic acid in the presence of an activating agent such as carbonyl diimidazole or a dialkyl carbodiimide; acid catalysis under dehydrating conditions; or transesterification using an acyl ester.

28. The process of claim 25, wherein the base is selected from one or more of tertiary amines, such as triethylamine or diisoproylethylamine; or an aromatic amine base, such as pyridine or imidazole, optionally in the presence of a catalytic stronger base such as dimethylaminopyridine.

29. The process of claim 25, wherein the suitable solvent is selected from one or more cyclic or acyclic ethereal solvents, such as 1,4-dioxane, 2-methyl tetrahydrofuran or tertiary butyl methyl ether; an ester solvent, such as ethyl acetate or isopropyl acetate; an aromatic solvent such as toluene.

30. The process of claim 25, wherein the suitable temperature is from -5° C. to 40° C.

31. The process of claim 25, wherein the addition of the acetylating agent optionally in the suitable solvent is performed over a period of 30 minutes, such as more than 3 hours.

32. The process of claim 25, wherein the reaction time is at from 1 to 24 hours.

33. The process of claim 25, comprising a preceding step for preparing the compound having formula IX wherein the preceding step comprises reacting a compound of formula VIII 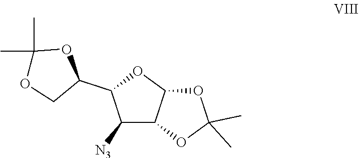
with an acid in a suitable solvent at a suitable temperature for a sufficient reaction time, for preparing the compound of formula IX.

34. The process of claim 33, wherein the acid is selected from one or more acidic cation exchange resin, such as Amberlite IR-120 H, dilute hydrochloric acid, or p-toluene sulfonic acid.

35. The process of claim 33, wherein the suitable solvent is a mixture of an organic solvent and water, such as 1,4-dioxane, 2-methyl tetrahydrofuran, tetrahydrofuran, or acetonitrile and water.

36. The process of claim 33, wherein the suitable temperature is from 25° C. to 70° C.

37. The process of claim 33, wherein the reaction time is 1 to 24 hours.

38. The process of claim 33, comprising a preceding step for preparing the compound having formula VIII wherein the preceding step comprises reacting a compound of formula VII 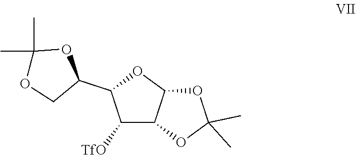
with a suitable azide in a suitable solvent at a suitable temperature for a sufficient reaction time, for preparing the compound of formula VIII.

39. The process of claim 38, wherein the azide is selected from an azide salt, such as sodium azide or potassium azide.

40. The process of claim 38, wherein the suitable solvent is selected from one or more dipolar aprotic solvents, such as dimethylformamide, dimethylsulfoxide or acetonitrile; or biphasic systems, such as tertiary butyl methyl ether or similar water immiscible solvents, such as 2-methyl tetrahydrofuran/water with a phase transfer catalyst, such as tetrabutylammonium bromide.

41. The process of claim 38, wherein the suitable temperature is from 0° C. to

42. The process of claim 38, wherein the reaction time is 30 min to 22 hours.

43. The process of claim 38, comprising a preceding step for preparing the compound having formula VII wherein the preceding step comprises reacting a compound of formula VI 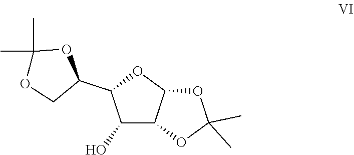
with a suitable base in a suitable solvent followed by a triflating agent in a suitable solvent at a suitable temperature for a sufficient reaction time, for preparing the compound of formula VII.

44. The process of claim 43, wherein the suitable base is pyridine or a hindered aliphatic tertiary amine, such as diisopropylethylamine.

45. The process of claim 43, wherein the triflating agent is selected from one or more trifluoromethanesulfonic anhydride or an equivalent triflating agent, such as N-phenyl-bis (trifluoromethanesulfonimide).

46. The process of claim 43, wherein the suitable solvent is independently selected from an aprotic solvent, such as tertiary butyl methyl ether, toluene or tetrahydrofuran.

47. The process of claim 43, wherein the suitable temperature is from −5° C. to 30° C.

48. The process of claim 43, wherein the reaction time is at least 1 hour.
